(19) United States
(12) Patent Application Publication
(10) Pub. No.: US 2016/0143569 A1
(43) Pub. Date: May 26, 2016

(54) SYSTEM TO IMPROVE THE QUALITY OF THE BLOOD SAMPLE OBTAINED FROM INFANTS FEET AND DSIPOSABLE HEATABLE BOOTY THEREOF

(71) Applicant: Jacqueline Ivonne Santos Rodriguez, Maldonado (UY)

(72) Inventor: Jacqueline Ivonne Santos Rodriguez, Maldonado (UY)

(21) Appl. No.: 15/015,210

(22) Filed: Feb. 4, 2016

Related U.S. Application Data
(63) Continuation-in-part of application No. 13/904,137, filed on May 29, 2013, now abandoned.
(60) Provisional application No. 61/652,480, filed on May 29, 2012.

(51) Int. Cl. A61B 5/15; A43B 3/30; A43B 7/02; A61B 5/151

(52) U.S. Cl. CPC A61B 5/150076; A61B 5/150412; A61B 5/150358; A61B 5/15105; A43B 3/30; A43B 7/02; A61B 2503/045

(57) ABSTRACT

The present invention relates to methods used to improve a quantity and a quality of a blood sample obtained from a heel of a foot of a newborn baby, particularly as sample for the neonatal screening.

### CROSS REFERENCE TO RELATED U.S. APPLICATION

This application is a continuation-in-part of patent application Ser. No. 13/904,137 filed May 29, 2013, entitled, SYSTEM TO IMPROVE THE QUALITY OF THE BLOOD SAMPLE OBTAINED FROM INFANTS FEET AND DISPOSABLE HEATABLE BOOTY THEREOF, pending, which claimed priority over U.S. provisional Application No. 61/652,480 filed May 29, 2012, the disclosures of both applications are incorporated herein by reference.

### FIELD OF THE INVENTION

The present invention relates to methods used to improve a quantity and a quality of a blood sample obtained from a heel of a foot of a newborn baby, particularly as sample for the neonatal screening.

### BACKGROUND OF THE INVENTION

In many third world countries, the so-called mandatory neonatal research has been implanted; in other countries of the world this neonatal research is voluntary. It is a method of early diagnosis of diseases, which applies especially in newborn children of forty hours old.

The neonatal screening is a method of early diagnosis of many diseases, such as phenylketonuria or hyperphenylalaninemia, adrenal hyperplasia, hypothyroidism, which can cause in the newborn mental retardation, among others, but if detected and treated early and at the appropriate time would not lead to major problems. As research progresses, it is discovered that other diseases may be detected early using this diagnosis method.

This method of diagnosis involves, in many countries, the heel puncture of the newborn, from where samples of blood are extracted, by using a Lancet.

For the correct sampling during these procedures, it is necessary for the blood to go out of the body fluidly; it is well known that the use of the Lancet does not provide the security that the blood-goes out of the body fluidly. In some regulations and instructions that are provided to the person taking the samples, they are encouraged to rub or massage the newborn's heel prior to the puncture. It is also possible to see that some of the people who take the samples they press the heel of the newborn in search of better blood samples. The most frequent is to see the person taking the samples to repeat the punctures several times in order to obtain a proper sample.

The pressure on the delicate newborn's heel may cause rupture of capillaries, hemorrhage, rupture of cartilage, contamination of the blood sample with interstitial and intracellular fluids. Rubbing or massaging the heel may cause hemolysis and other injuries that may compromise the quality of subsequent samples, in case there is a need to repeat them. Multi-puncture may be a cruel procedure resisted by the newborn baby and his/her relatives, making more difficult work for the person taking the sample.

It is well known that warming up the skin in the area to be punctured increases the capillary action by vessel dilation; this increases blood flow in the area to be punctured. Academic work, at the international level, discusses the need to warm the infant's heel in order to facilitate obtaining the blood sample.

Many devices are known in the form of bandages or disposable cloths that allow to warm the children's heels prior to the puncture. These bandages or disposable cloths can be heated, others are made of thermal materials; these cloths or bandages are applied to the area that needs to be heated and others are wrapped around the feet or ankles, or have other adjustment mechanisms. In the case of babies, if these bandages or cloths are heated in excess they can cause burns on the delicate skin of the babies, and with regards to its shape, the baby can get rid of them, because they are uncomfortable or annoying. This further complicates the work of the person taking the sample and makes necessary to perform multi-puncture, since its use does not guarantee the proper capillary dilation in the infant foot, so the quality of the samples it is not the sought one.

Also, there are known heaters, including fragments or sectors, that are joined to each other by tape or other adjustment elements that are placed around the foot or the heel; these devices need brooches that engage into holes to avoid the patient removing them or the separation or movement of the fragments and do not fulfill its function properly. These devices are not suitable for babies, since they constantly move, which complicates its placement and on the other hand, they may be uncomfortable so the babies may try to remove them. The result is the same as in the case of bandages or cloth, that is the multi-puncture to achieve the quality looked for in the required blood samples. On the other hand, the fact that its making is complicated increases the production cost which can tempt to reuse them instead of discarding them because they are usually contaminated with fluids of patients.

To solve these problems, in the U.S. provisional application No. 61/652,480, the inventor proposed a disposable heatable booty for heating the babies' heels prior to capillary puncture.

### OBJECT OF THE INVENTION

It is an object of the present invention to prevent the multi-puncture of the newly born, and, in general, to decrease the number of repetitions of the puncture of the newborn's heel, through providing a system to improve the quality and quantity of the blood sample, achieving efficiency.

It is another object of the invention to help achieve an early diagnosis of diseases and treatment at the proper time, if necessary.

A third object of the invention is to implement a user-friendly device for the newborn baby and their families, disposable and heatable, which facilitates the taking of the blood sample for the neonatal research and other studies that require a skin puncture. It is well known that the booty shape is more comfortable and friendly for babies since booties are one of the elements that are worn by them. On the other hand, family members do not provide resistance to place booties to bring warmth to the delicate feet of the baby.

It is a fourth object of the invention to achieve an adequate vasodilation and a greater blood supply by warming the foot of the child, especially newborns.

A fifth object of this invention is to reduce the suffering of the child during the execution of the procedure of obtaining the blood sample and improve the quality of the health service. The use of bags of hot water, microwave heatable bags, heatable bandages, rubbing, or massaging, can cause injury and burns on the delicate skin of the newborn and in other cases lesions on their cartilage, bleeding, among other possible problems.

It is a sixth object of this invention to simplify the work of the nursing staff responsible for obtaining the blood samples.

It is a seventh object of this invention to reduce the costs of necessary materials.

### BRIEF DESCRIPTION OF DRAWINGS

FIG. 1 shows a perspective front view a disposable heatable booty for capillary puncture according to the present invention;

FIG. 2 shows a front view of the disposable heatable booty of FIG. 1;

FIG. 3 shows a side view of the disposable heatable booty of FIG. 1 illustrating an upper section which corresponds to the elastic cuff that adjusts to the ankle C of the newborn baby, the rear part D, the front part E. and the bottom part F;

FIG. 4 shows a cross-sectional view along line B-B″ of FIG. 2 illustrating the booty (1) made of paper fabric, the cuff (2) of the booty made of elastic fabric, sewing of the cuff (3) that it connects to the body of the booty, gel capsule (7) with seeds (9), soft fabric (8) that surround the gel capsule, part of the booty that goes on the instep of the foot (6);

FIG. 5 shows a cross-sectional view along line A-A″ of FIG. 2 showing the booty (1) made of paper fabric, cuff (2) in booty made of elastic fabric, cuff's sewing (3), uppers or upper part (5) of the booty, seam (4) that joins the shoe upper to the other part of the booty, the gel capsule (7) with seeds (9), and soft fabric (8) that surrounds the gel capsule;

FIG. 6 shows a cross-sectional view B-B″ illustrating the booty (1), the cuff (2) made of elastic fabric, the sewing (3) of the cuff, the upper or upper part (5) of the booty, the gel capsule (7) with seeds (9), the soft fabric (8) that envelops the gel capsule;

FIG. 7 shows a cross-sectional view of the bottom section of the booty (1) illustrating the interior of the gel capsule (7) including the seeds (9) located on its interior;

FIG. 8 shows a cross-sectional view B-B″ illustrating the booty (1), cuff (2) made of elastic fabric, sewing (3) of the cuff, the portion of the booty (6) that goes on the instep of the foot, seeds (9), an insert (11) made of paper fabric, soft fabric (10) that surrounds the insert and seeds;

FIG. 9 shows a cross-sectional view A-A″ illustrating the booty (1), cuff (2) made of elastic fabric , sewing (3) of the cuff, uppers or upper part (5) of the booty, seam (4) that joins the shoe upper to the other part of the booty, seeds (9), insert (11) made of paper fabric, soft fabric (10) that surrounds template and seeds;

FIG. 10 shows a cross-sectional view B-B″ showing the booty (1), the cuff (2) made of fabric elastic, upper or upper part (5) of the booty, seeds (9), sole (11) made of paper fabric, soft fabric (10) that surrounds the sole and the seeds;

FIG. 11 shows a gel capsule (7) having inside the seeds (9), a soft fabric (8) surrounds the gel capsule;

FIG. 12 shows the insert (11) made of paper fabric, seeds (9) placed on top of the insert, and a soft fabric (10) that surrounds the insert and the seeds;

FIG. 13 shows the area (12) of the bottom of the foot of the newborn pointing the puncture zone where the user may puncture the heel of the newborn;

FIG. 14 shows a filter paper (13) where blood samples are being deposited showing the blood drops (14) obtained without using the booty according to the present invention; and

FIG. 15 shows a filter paper (13) where blood drops (15, 16) are being deposited after the use of the booty according to the present invention, as can be observed, the blood drops (15, 16) are larger in size than the blood drops (14) of FIG. 14.

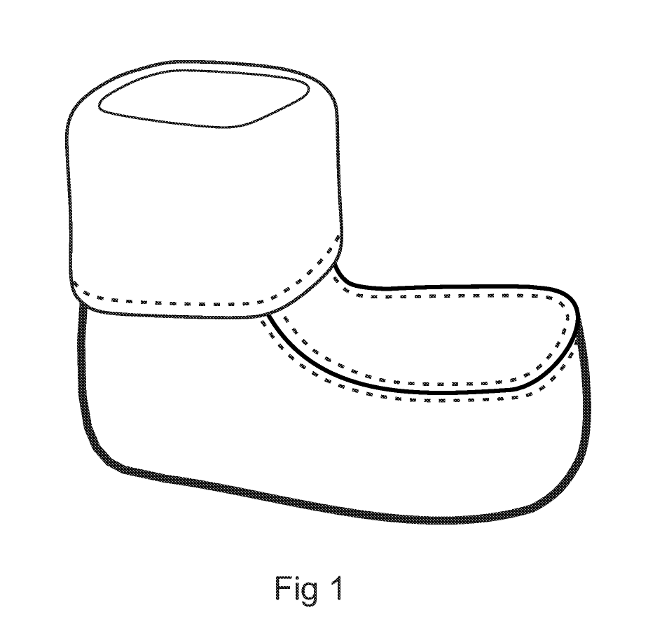

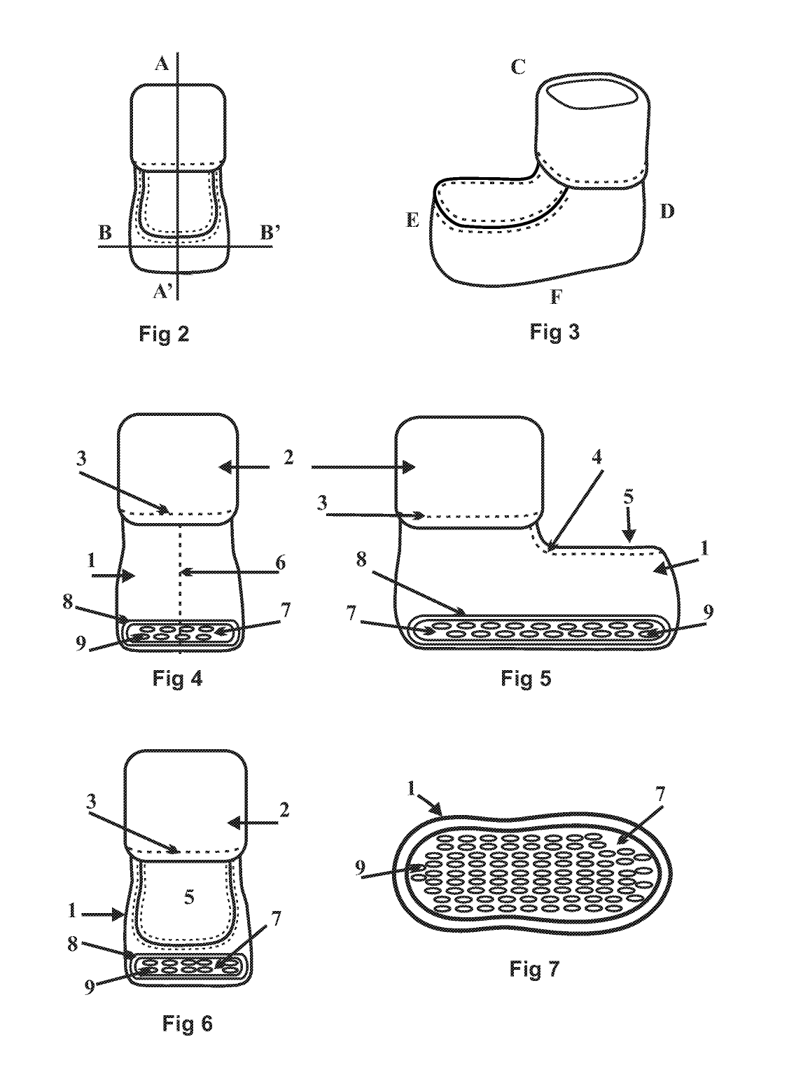

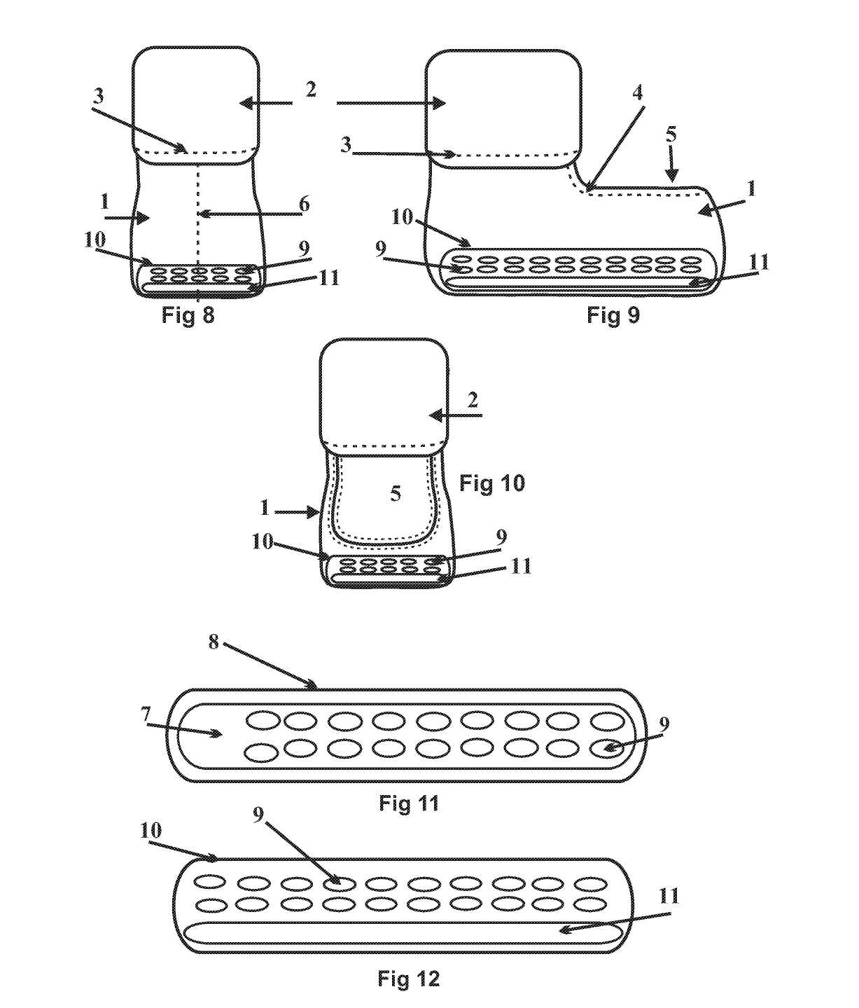

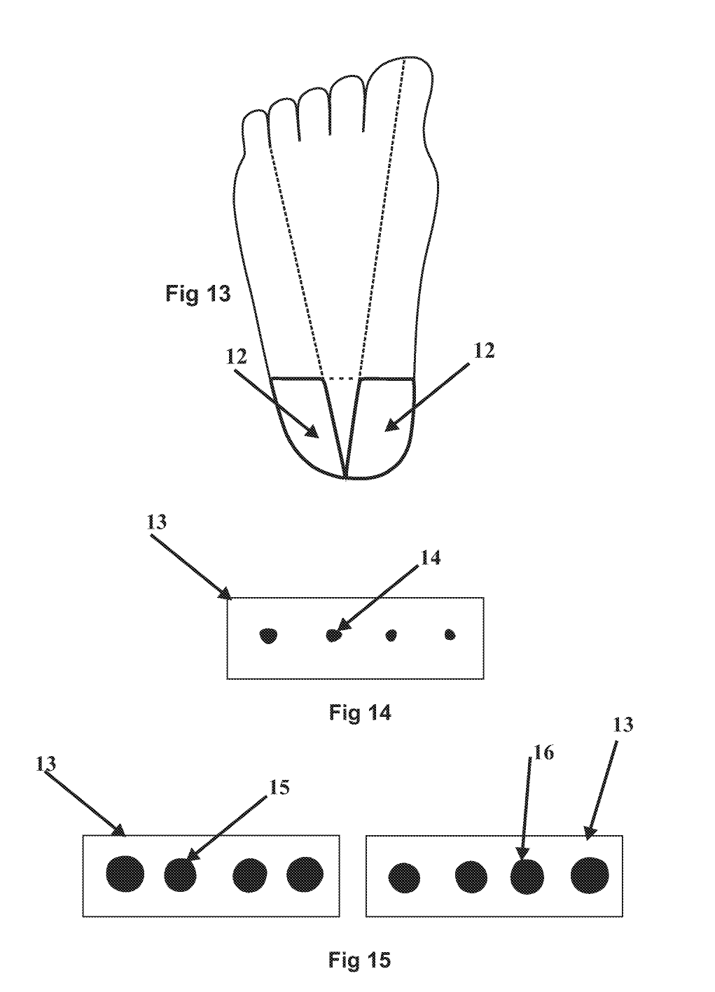

### DESCRIPTION OF THE INVENTION

The present invention relates to a method to improve a quantity and a quality of a blood sample from a heel of a foot of a newborn baby additionally enabling the easier and more comfortable blood collection and dropping on a screening book as blood sample for the neonatal screening.

The method comprises the steps of:

providing a disposable heatable booty,

warming a disposable heatable booty in a warming device,

placing the heated disposable booty in a newborn baby foot,

warming the foot of the newborn baby obtaining an adequate vasodilation in a heel of the newborn baby,

removing the booty from the newborn baby foot after a predetermined time,

puncturing the heel of the newborn baby foot with a lancet,

pressing the heel of the newborn baby foot manually to obtain a blood sample,

letting newborn blood sample drops from the heel of newborn baby foot and be deposited on a filter paper which will be attached to the screening book.

The testing of the prototypes demonstrate that blood samples improve quantity and quality when using a disposable heatable booty. The blood drops are larger and the risk of contaminating the samples during the blood collection decreases due to better peripheral dilation of the blood vessels and easier manipulation of the booty in relation to other implements as for instance warm wet bandages or proceedings as for instance frictions.

A sterile disposable heatable booty to bring warmth to the foot of the newborn for the realization of the capillary puncture.

The disposable and heatable booty according to the present invention includes:

a body, the body including an upper section, a lower section, a bottom section, and an interior section, the lower section of the body is designed to entirely cover the foot of the newborn baby, the booty is made of a disposable heatable, and soft material selected from the group consisting of paper fabric, micro fleece fabric, polar fleece fabric, cotton fabric, vinyon fabric, flannel fabric;

a cuff secured to the upper section of the body, the cuff is designed to cover an ankle of the newborn baby, the cuff is made of a stretch heatable fabric that adjusts to the ankle of the newborn baby, the stretched heatable fabric may be, for example an elastic, aramid fabric, thermoplastic polyurethane fabric, or vinyon fabric,

an insert placed on the interior section of the body at the bottom section of the heatable booty,

seeds placed over the insert, the seeds are heatable; and

a cover completely surrounding the insert and seeds.

The booty is made of a soft fabric, that is disposable and heatable for example, paper fabric, micro fleece fabric, polar fleece fabric, cotton fabric, vinyon fabric, or flannel fabric.

The cuff is secured to the upper section of the body, the cuff is designed to cover an ankle of the newborn baby, the cuff is made of an stretch fabric capable to return to its normal shape spontaneously after a distortion. The stretch fabric adjusts to the ankle of the newborn baby. The stretch fabric may be, for example, elastic, aramid fabric, thermoplastic polyurethane fabric, spandex fabric, lycra fabric, or vinyon fabric.

The heatable booty according to the present invention may be a single piece booty or may have the body and the cuff attached to each other. The booty covers the bottom of the foot, sides, the instep of the foot, and the toes. The body of the booty can be sewn or attached by other means to a cuff made of elastic fabric for example, aramid fabric, thermoplastic polyurethane fabric or vinyon fabric. The cuff gently adjusts to the ankle area of the baby. So the foot is completely covered. The cuff gently adjusts the body of the booty to the infant ankle to avoid the infant from removing it with its natural movements.

During the testings of the prototype, it was demonstrated to provide efficiency:

to place inside the booty an insert made of, for example, paper fabric, micro fleece fabric, polar fleece fabric, cotton fabric, vinyon fabric, flannel fabric, at the bottom of it,

to place heatable seeds of, for instance, wheat seeds, lavender seeds, linen seeds, over the insert,

to wrap the insert with the heatable seeds inside a cover made of a soft cloth, for instance, polar fleece fabric, cotton fabric or flannel fabric in order to prevent burns in the delicate skin of the newborn.

The insert with heatable seeds and the cover ay be loose or tight to the bottom of the body of the booty.

The testings have revealed that it is important that the heatable seeds are placed over the insert, not in its interior, because it is easier for the heat to reach a newborn foot.

The seeds used on the present invention need to promote analgesia and anti-inflammatory properties in order to promote the vasodilatation. In addition, it is important that the heatable seeds to be used in the present invention have the property of keeping warm. The seeds use by the present invention may be, for example, wheat seeds, lavender seeds, and linen seeds.

The insert with the heatable seeds were wrapped inside the cover and then placed in the interior section of the booty. Then, the booties were heated in a microwave oven during less than 3 minutes.

The booties can be heated in a microwave oven or other devices that may be researched and developed to warm them and that allow a control of the temperature to be applied.

The inserts with the seeds wrapped inside a cover can be heated in a microwave oven or other devices that may be researched and developed to warm them and that allow a control of temperature to be applied.

The booties should not be connected to an electrical power cord or electricity grid since is important to carefully control the temperature to avoid burns to the newborn.

In addition, the booties should be free of chemical substance that allows self-heating as a way to provide safety to the newborns.

After heated, the booties are placed at the feet of the baby or child transmitting heat to the heels producing peripheral dilation of the blood vessels. With the heat, the skin becomes red, since capillaries of the skin are dilated to increase the heat exchange area with the surface area, and as a consequence increases the blood supply.

In another embodiment, the insert with heatable seeds may be replace by a thermal gel capsule with heatable seeds in its interior. In case of replacing the insert by a thermal gel capsule with heatable seeds in its interior, the inventor recommends to use the same type of seeds that is to say wheat, lavender or linen. The thermal gel capsule with heatable seeds in its interior should be wrapped in a cover of soft cloth as for instance, polar fleece fabric, cotton fabric or flannel fabric.

The baby's skin does not come in contact with the seeds, but it comes in contact with the soft fabric of the wrapping.

The booty measures that have proven to be appropriate during the testing of the prototype are the following: 7 cm long, 4 cm wide, 5 cm high, and weighs approximately 15 grams.

The measurement of the insert with heatable seeds wrapped in soft fabric is proportional to the above indicated measurements.

It is not recommended to use the commercially available bags with seeds which are normally for heating, for example, babies cribs, since the air between the seeds inside the bag interrupts the heat transmission between them, causing a heat loss, and minimizing the total volume of the condensate element of heat that are the seeds. In addition the use of bag containing seeds in its interior do not allow to have a precise control of the temperature, which is a very important factor when working with the delicate skin of a newborn.

The use of wheat, lavender or linen seeds gives an additional property, these types of seeds are proper to keep warm so the inserts or thermal gel capsules can be kept warm after removing from microwave oven.

During the testing of the prototypes at the newborns' feet, highly satisfactory results were found: effective heating of the newborn's heel; injuries do not occur in the skin unlike what happens with the friction of the same; there are no burns; the blood samples significantly improve in quantity and quality; the babies and their families do not resist and, therefore, allow the trained personnel to work comfortably and efficiently.

### Use of the Booty for Blood Sampling

In an example of use during the testing of the prototype, the booty is heated approximately 1 minute in the microwave device, the booty is placed on the foot of the newborn to be punctured and it is necessary to wait for between 10 and 20 minutes, while the mother nurses the baby if the extraction is destined to the neonatal research sampling, since in this case it is important that the baby has eaten food containing protein (breast milk, supplement with protein content).

The person taking the sample cleans his/her hands and makes sure to have the following materials available: alcohol, cotton, gloves, Lancet, leuco tape, support for drying of sample, and filter paper for deposit of the sample.

The person taking the sample ask the person who has the newborn in her arms to place the newborn in an upright position and remove the booty, the person taking the sample discards the booty into a container for disposable materials. The person taking the sample proceeds to perform the asepsis to all of the bottom part of the newborn's foot. The person taking the sample punctures the heel with a Lancet on the puncture area, cleans the heel to discard the interstitial fluid which arises immediately after the puncture, and leaves to form the blood drop completely. Takes the paper intended for the raking of the blood sample in such way that the end of the blood drop makes contact with it and allows to spread. The person taking the sample ensures that the drop went through the filter paper on both sides. The person taking the sample repeats the procedure to obtain at least three drops. The filter paper is allowed to dry between 4 and 6 hours.

If for any reason it is necessary to repeat the puncture. The person taking the sample uses another disposable heatable booty by repeating the procedure.

What is claimed is:

1. A method to improve a quantity and a quality of a blood sample obtained from a heel of a foot of a newborn baby comprising:
providing a disposable heatable booty, the heatable booty including an insert located on a bottom side of the booty and heatable seeds located over the inset, and a cover completely surrounding the insert and heatable seeds;
warming the entire disposable heatable booty including the insert, heatable seeds, and cover in a warming device;
placing the heated disposable booty in the foot of the newborn baby;
removing the booty from the foot of the newborn baby;
puncturing the heel of the newborn baby foot with a lancet;
pressing the heel of the foot of the newborn baby;
letting newborn blood sample drops from the heel of the newborn baby and be deposited on a filter paper;
attaching the filter paper to a screening reader.

2. A disposable heatable booty designed to fit on a foot of a newborn baby comprising:
a body, the body including an upper section, a lower section, a bottom section, and an interior section, the lower section of the body is designed to entirely cover the foot of the newborn baby, the booty is made of a disposable, heatable, and soft material selected from the group consisting of paper fabric, micro fleece, and polar fleece fabric, cotton fabric, vinyon fabric, flannel fabric,
a cuff secured to the upper section of the body, the cuff is designed to cover an ankle of the newborn baby, the cuff is made of a stretchable and heatable fabric selected from the group consisting of an elastic fabric, an aramid fabric, a thermoplastic polyurethane fabric, or a vinyon fabric, the cuff adjusts to the ankle of the newborn baby;
an insert made of disposable heatable fabric selected from the group consisting of a paper fabric, a micro fleece fabric, a polar fleece fabric, a cotton fabric, a vinyon fabric, or a flannel fabric, the insert is placed on the interior bottom section of the body;
heatable seeds placed over the insert, the heatable seeds are selected from the group consisting of wheat seeds, lavender seeds, and linen seeds; and
a cover completely surrounding the insert and the heatable, the cover being made of polar fleece fabric, cotton fabric, or flannel fabric.

3. The disposable heating booty according to claim 2, wherein the disposable heatable booty is made of a one piece fabric.

4. The disposable heating booty according to claim 2, wherein the body and the cuff are made of two different pieces of fabric sewn together.

5. The disposable heating booty according to claim 2, wherein the insert and the heatable seeds are replaced by a thermal gel capsule having heatable seeds inside.

6. A method to improve a quality and a quantity of blood as sample for the neonatal screening, obtained from a foot of a newborn baby comprising:
a) providing a disposable heatable booty designed to fit the foot of the newborn baby and including: body, the body including an upper section, a lower section, a bottom section, and an interior section, the lower section of the body is designed to entirely cover the foot of the newborn baby, the booty is made of a disposable, heatable, and soft material selected from the group consisting of paper fabric, micro fleece, and polar fleece fabric, cotton fabric, vinyon fabric, flannel fabric,
a cuff secured to the upper section of the body, the cuff is designed to cover an ankle of the newborn baby, the cuff is made of a stretchable and heatable fabric selected from the group consisting of an elastic fabric, an aramid fabric, a thermoplastic polyurethane fabric, or a vinyon fabric, the cuff adjusts to the ankle of the newborn baby;
an insert made of disposable heatable fabric selected from the group consisting of a paper fabric, a micro fleece fabric, a polar fleece fabric, a cotton fabric, a vinyon fabric, or a flannel fabric, the insert is placed on the interior bottom section of the body;
heatable seeds placed over the insert, the heatable seeds are selected from the group consisting of wheat seeds, lavender seeds, and linen seeds; and
a cover completely surrounding the insert and the heatable, the cover being made of polar fleece fabric, cotton fabric, or flannel fabric
b) heating the booty including the inset and seed in a warming device;
c) placing the heated disposable booty in the foot of the newborn baby;
d) removing the booty from the foot of the newborn baby;
e) puncturing the heel of the foot of the newborn baby with a lancet;
f) pressing the heel of the foot of the newborn baby;
g) letting newborn blood sample drops from the heel of the newborn baby and be deposited on a filter paper; and
h) attaching the filter paper to a screening reader.

8. The method according to claim 7, wherein the disposable heatable booty is made of a one piece fabric.

9. The method according to claim 7, wherein the body and the cuff are made of two different pieces of fabric sewn together.
